(19) United States
(12) Patent Application Publication
(10) Pub. No.: US 2016/0159868 A1
(43) Pub. Date: Jun. 9, 2016

(54) PRODUCTION OF SOY PROTEIN PRODUCTS WITH REDUCED ASTRINGENCY (II)

(71) Applicant: Burcon NutraScience (MB) Corp., Winnipeg (CA)

(72) Inventors: Martin Schweizer, Winnipeg (CA); Kevin I. Segall, Winnipeg (CA)

(73) Assignee: Burcon NutraScience (MB) Corp., Winnipeg (CA)

(21) Appl. No.: 14/872,430

(22) Filed: Oct. 1, 2015

Related U.S. Application Data
(60) Provisional application No. 62/058,159, filed on Oct. 1, 2014.

(51) Int. Cl. C07K 14/415; A23L 2/66; C07K 1/36

(52) U.S. Cl. CPC C07K 14/415; C07K 1/36; A23L 2/66

(57) ABSTRACT

The present invention is directed to soy protein products of reduced astringency. The reduced astringency soy protein products of the present invention may be obtained by using membrane processing to fractionate soy protein solutions, which provide soy protein products which are completely soluble and heat stable in aqueous media at acid pH value of less than about 4.4, into lower molecular weight, less astringent proteins and higher molecular weight, more astringent proteins.

### FIELD OF THE INVENTION

The present invention relates to novel and inventive soy protein products, preferably soy protein isolates and novel and inventive methods for the production thereof. More particularly, the present invention relates to soy protein products of reduced astringency.

### BACKGROUND TO THE INVENTION

In U.S. patent application Ser. No. 12/603,087 filed Oct. 21, 2009 (now U.S. Pat. No. 8,691,318), Ser. No. 12/923,897 filed Oct. 13, 2010 (now U.S. Pat. No. 8,563,071), Ser. No. 13/879,418 filed Aug. 1, 2013 (published as US Patent Application Publication No. 20130316069), assigned to the assignee hereof and the disclosures of which are incorporated herein by reference, there is described the provision of soy protein products having a protein content of at least about 60 wt % (N×6.25) on a dry weight basis (d.b.), preferably soy protein isolates having a protein content of at least about 90 wt % (N×6.25) d.b. These soy protein products have a unique combination of properties, namely:
completely soluble in aqueous media at acid pH values of less than about 4.4;
heat stable in aqueous media at acid pH values of less than about 4.4;
does not require stabilizers or other additives to maintain the protein product in solution;
is low in phytic acid; and
requires no enzymes in the production thereof.

In addition, these soy protein products have no beany flavour or off odours characteristic of other soy protein products.

These novel and inventive soy protein products are prepared by methods which comprise:
(a) extracting a soy protein source with an aqueous calcium salt solution, preferably an aqueous calcium chloride solution to cause solubilization of soy protein from the protein source and to form an aqueous soy protein solution,
(b) separating the aqueous soy protein solution from residual soy protein source,
(c) optionally diluting the aqueous soy protein solution,
(d) adjusting the pH of the aqueous soy protein solution to a pH of about 1.5 to about 4.4, preferably about 2 to about 4, to produce an acidified clear soy protein solution,
(e) optionally concentrating the acidified clear soy protein solution while maintaining the ionic strength substantially constant by a selective membrane technique,
(f) optionally diafiltering the optionally concentrated soy protein solution, and
(g) optionally drying the optionally concentrated and optionally diafiltered soy protein solution.

These soy protein products preferably are isolates having a protein content of at least about 90 wt %, preferably at least about 100 wt % (N×6.25) d.b.

In certain acidic beverages, particularly those having a pH at the low end of the acceptable pH range for acidic beverages, these soy protein products may, in some cases, induce an astringent sensation in the mouth.

### SUMMARY OF THE INVENTION

It has now been determined by the present inventors, and first disclosed in the present application and in the application from which the present application claims priority, that this astringency can be reduced or eliminated by modifying the procedure used to manufacture the soy protein products. Less astringent proteins appear to be of lower molecular weight than the more astringent species. Without wishing to be bound by theory, the proteins of higher molecular weight may interact with salivary proteins to induce astringency and thus their removal may thereby provide a less astringent product.

In accordance with an aspect of the present invention, the more astringent protein component is separated from the less astringent protein component by membrane processing. Concentration and/or diafiltration of a protein solution containing a mixture of the more and less astringent proteins using a membrane with an appropriate pore size allows the smaller, less astringent proteins to pass through with the permeate, while retaining the more astringent species in the retentate. The less astringent proteins may be separated from contaminants by a subsequent concentration and/or diafiltration step using a membrane having a smaller pore size than that employed in the fractionation step. The purified less astringent protein fraction is a product of the present invention.

In accordance with another aspect of the present invention, there is provided a method of preparing soy protein product with reduced astringency when tasted in aqueous solution at a pH below about 5, which method comprises:
(a) extracting a soy protein source with an aqueous calcium salt solution to cause solubilization of soy protein from the protein source and to form an aqueous soy protein solution,
(b) separating the aqueous soy protein solution from residual soy protein source,
(c) optionally diluting the aqueous soy protein solution,
(d) adjusting the pH of the aqueous soy protein solution to a pH of about 1.5 to about 4.4 to produce an acidified soy protein solution,
(e) optionally clarifying the acidified soy protein solution if it is not already clear,
(f) alternatively from steps (b) to (e), optionally, diluting and then adjusting the pH of the combined aqueous soy protein solution and residual soy protein source to a pH of about 1.5 to about 4.4 and then separating the acidified, preferably clear, soy protein solution from residual soy protein source, and
(g) fractionating the proteins in the acidified soy protein solution of step (e) or (f) to separate lower molecular weight, less astringent proteins from higher molecular weight, more astringent proteins, wherein said fractionation step is effected by membrane processing the acidified aqueous soy protein solution to fractionate the protein components of the acidified aqueous soy protein solution into a higher molecular weight fraction in a first retentate and a lower molecular weight fraction in a first permeate,
(h) membrane processing the first permeate to retain the lower molecular weight fraction protein components in a second retentate and to permit contaminants to pass the membrane in a second permeate, and
(i) optionally drying the second retentate to provide a soy protein product of reduced astringency.

In an embodiment of the present invention, the fractionation step (g) is effected by partially concentrating or concentrating and/or diafiltering the acidified aqueous soy protein solution to provide a first retentate and a first permeate.

In an embodiment of the present invention, the acidified protein solution is partially concentrated to provide a first retentate having a protein concentration of less than about 50 g/L or fully concentrated to provide a first retentate having a protein concentration of about 50 to about 300 g/L. In another embodiment of the present invention, the acidified protein solution is partially concentrated to provide a first retentate having a protein concentration of less than about 50 g/L or fully concentrated to provide a first retentate having a protein concentration of about 100 to about 200 g/L.

In an embodiment of the present invention, diafiltration is applied to the acidified protein solution or partially concentrated acidified protein solution and the diafiltered solution is further concentrated to provide a first retentate having a protein concentration of about 50 to about 300 g/L. In another embodiment of the present invention, diafiltration is applied to the acidified protein solution or partially concentrated acidified protein solution and the diafiltered solution is further concentrated to provide a first retentate having a protein concentration of about 100 to about 200 g/L.

In an embodiment of the present invention, the diafiltration step is effected using about 1 to about 40 volumes of diafiltration solution. In another embodiment of the present invention, the diafiltration step is effected using about 2 to about 25 volumes of diafiltration solution.

In an embodiment of the present invention, the diafiltration solution is water, acidified water, a dilute saline solution or an acidified dilute saline solution. In an embodiment of the present invention, the saline solution is selected from the group consisting of sodium chloride, calcium chloride and combinations thereof.

In an embodiment of the present invention, the membrane processing step (g) is effected by microfiltration using membranes having a pore size selected from the group consisting of about 0.02 to about 0.1 μm and about 0.08 to about 0.1 μm or by ultrafiltration using membranes having a molecular weight cut-off selected from the group consisting of about 5,000 to about 1,000,000 daltons and about 50,000 to about 1,000,000 daltons.

In an embodiment of the present invention, the membrane processing of the first permeate in step (h) is effected by partially concentrating or concentrating and/or diafiltering the first permeate to provide a second retentate and a second permeate.

In an embodiment of the present invention, the first permeate is partially concentrated to provide a second retentate having a protein concentration of less than about 10 g/L or concentrated to provide a second retentate having a protein concentration selected from the group consisting of about 10 to about 300 g/L and about 100 to about 200 g/L.

In an embodiment of the present invention, diafiltration is applied to the first permeate or partially concentrated first permeate and the diafiltered solution is further concentrated to provide a second retentate having a protein concentration of about 10 to about 300 g/L. In another embodiment of the present invention, diafiltration is applied to the first permeate or partially concentrated first permeate and the diafiltered solution is further concentrated to provide a second retentate having a protein concentration of about 100 to about 200 g/L.

In an embodiment of the present invention, the diafiltration step is effected using about 1 to about 40 volumes of diafiltration solution. In another embodiment of the present invention, the diafiltration step is effected using about 2 to about 25 volumes of diafiltration solution.

In an embodiment of the present invention, the diafiltration solution is water, acidified water, a dilute saline solution or an acidified dilute saline solution. In an embodiment of the present invention, the saline solution is selected from the group consisting of sodium chloride, calcium chloride and combinations thereof.

In an embodiment of the present invention, the diafiltration operation is effected until no significant further quantities of contaminants or visible colour are present in the second permeate, or until the second retentate has been sufficiently purified so as, when dried to provide a soy protein isolate having a protein content of at least about 90 wt % (N×6.25) d.b.

In an embodiment of the present invention, the membrane processing step is effected by ultrafiltration using membranes having a molecular weight cut-off of about 1,000 to about 100,000 daltons. In another embodiment of the present invention, the membrane processing step is effected by ultrafiltration using membranes having a molecular weight cut-off of about 1,000 to about 10,000 daltons.

In accordance with another aspect of the present invention, there is provided a soy protein product having a protein content of at least about 60 wt % (N×6.25) d.b. and which
is completely soluble in aqueous media at acid pH values of less than about 4.4;
is heat stable in aqueous media at acid values of less than about 4.4;
does not require stabilizers or other additives to maintain the protein product in solution or suspension;
is low in phytic acid; and
is low in astringency when tasted in aqueous solution at a pH below about 5.

In an embodiment of the present invention, the process for the production of the soy protein product comprises the use of membrane processing for the fractionation of high molecular weight, high astringency proteins and low molecular weight, low astringency proteins.

In an embodiment of the present invention, no enzymes are utilized in the production of the soy protein product of the present invention.

In an embodiment of the present invention, the soy protein product has a protein content of at least about 90 wt % (N×6.25) d.b. In another embodiment of the present invention, the soy protein product has a protein content of at least about 100 wt % (N×6.25) d.b.

In an embodiment of the present invention, the soy protein product is not hydrolysed.

In an embodiment of the present invention, the soy protein product has a phytic acid content of less than about 1.5 wt %. In another embodiment of the present invention, the soy protein product has a phytic acid content of less than about 0.5 wt %.

In accordance with another aspect of the present invention, there is provided a soy protein product having a protein content of at least about 60 wt % (N×6.25) d.b., having low astringency when tasted in aqueous solution at a pH of below about 5 and which is substantially completely soluble in an aqueous medium at a pH of less than about 4.4.

In an embodiment of the present invention, the process for the production of the soy protein product comprises the use of membrane processing for the fractionation of high molecular weight, high astringency proteins and low molecular weight, low astringency proteins.

In an embodiment of the present invention, the soy protein product may be blended with water soluble powdered materials for the production of aqueous solutions of the blend. In an embodiment of the present invention, the water soluble powdered materials are a powdered beverage.

In an embodiment of the present invention, the soy protein product is in an aqueous solution. In an embodiment of the present invention, the aqueous solution is heat stable at a pH of less than about 4.4. In an embodiment of the present invention, the aqueous solution is a beverage. In an embodiment of the present invention, the beverage is a clear beverage in which the dissolved soy protein product is completely soluble and transparent in which the dissolved soy protein does not increase the cloud or haze level. In an embodiment of the present invention, the beverage is a non-transparent beverage in which the dissolved soy protein does not increase the cloud or haze level.

In accordance with another aspect of the present invention, there is provided a soy protein product having a molecular weight profile, which is:

about 0 to about 80% greater than about 100,000 Da;

about 0 to about 50% from about 15,000 to about 100,000 Da;

about 0 to about 35% from about 5,000 to about 15,000 Da; and

about 0 to about 20% from about 1,000 to about 5,000 Da.

In an embodiment of the present invention, the molecular weight profile is:

about 35 to about 75% greater than about 100,000 Da;

about 20 to about 45% from about 15,000 to about 100,000 Da;

about 2 to about 27% from about 5,000 to about 15,000 Da; and

about 1 to about 12% from about 1,000 to about 5,000 Da.

In accordance with another aspect of the present invention, there is provided a soy protein product having a molecular weight profile, which is:

about 32 to about 78% greater than about 100,000 Da;

about 18 to about 48% from about 15,000 to about 100,000 Da;

about 0 to about 31% from about 5,000 to about 15,000 Da; and

about 0 to about 17% from about 1,000 to about 5,000 Da.

In an embodiment of the present invention, the molecular weight profile is:

about 37 to about 73% greater than about 100,000 Da;

about 22 to about 43% from about 15,000 to about 100,000 Da;

about 2 to about 26% from about 5,000 to about 15,000 Da; and

about 1 to about 12% from about 1,000 to about 5,000 Da.

In an embodiment of the present invention, the molecular weight profile is determined by size exclusion chromatography at a pH of about 3.5. In another embodiment of the present invention, the molecular weight profile is determined by the method described in Example 19.

In an embodiment of the present invention, the protein content of the product is at least about 60 wt % (N×6.25) d.b. and the protein solubility at 1% protein w/v in water at a pH of about 2 to about 4 is greater than about 90%. In an embodiment of the present invention, the protein solubility at 1% protein w/v in water at a pH of about 5 to about 6 is greater than about 22%. In an embodiment of the present invention, the protein solubility at 1% protein w/v in water at a pH of about 7 is greater than about 90%. In an embodiment of the present invention, the protein solubility is determined by the protein method described in Example 3.

In an embodiment of the present invention, the protein content of the product is at least about 60 wt % (N×6.25) d.b. and the haze reading for a 1% protein w/v solution in water at a pH of about 4 is less than about 20%. In an embodiment of the present invention, the haze reading is determined by the method described in Example 4.

In an embodiment of the present invention, the protein content of the product is at least about 60 wt % (N×6.25) d.b., and the L* reading for a solution prepared by dissolving sufficient protein powder to supply 0.48 g of protein in 15 ml of water is greater than about 96.50. In an embodiment of the present invention, the b* reading for a solution prepared by dissolving sufficient protein powder to supply 0.48 g of protein in 15 ml of water is less than about 8. In an embodiment of the present invention, the b* reading for the dry protein powder is less than about 8.

In accordance with another aspect of the present invention, there is provided a soy protein product having a phytic acid content of less than about 1.5 wt % and having a molecular weight profile, which is:

about 7 to about 35% greater than about 100,000 Da;

about 21 to about 62% from about 15,000 to about 100,000 Da;

about 6 to about 28% from about 5,000 to about 15,000 Da; and

about 3 to about 28% from about 1,000 to about 5,000 Da.

In an embodiment of the present invention, the molecular weight profile is determined by size exclusion chromatography at a pH of about 6. In another embodiment of the present invention, the molecular weight profile is determined by the method described in Example 20.

In an embodiment of the present invention, the protein content of the product is at least about 60 wt % (N×6.25) d.b. and the protein solubility at 1% protein w/v in water at a pH of about 2 to about 4 is greater than about 90%. In an embodiment of the present invention, the protein solubility is determined by the protein method described in Example 3.

In an embodiment of the present invention, the protein content of the product is at least about 60 wt % (N×6.25) d.b. and the L* reading for a solution prepared by dissolving sufficient protein powder to supply 0.48 g of protein in 15 ml of water is greater than about 96.50. In an embodiment of the present invention, the b* reading for a solution prepared by dissolving sufficient protein powder to supply 0.48 g of protein in 15 ml of water is less than about 8. In an embodiment of the present invention, the b* reading for the dry protein powder is less than about 8.

In accordance with another aspect of the present invention, there is provided a soy protein product having a molecular weight profile, which is:

about 12 to about 30% greater than about 100,000 Da;

about 26 to about 57% from about 15,000 to about 100,000 Da;

about 11 to about 23% from about 5,000 to about 15,000 Da; and

about 8 to about 23% from about 1,000 to about 5,000 Da.

In an embodiment of the present invention, the molecular weight profile is determined by size exclusion chromatography at a pH of about 6. In another embodiment of the present invention, the molecular weight profile is determined by the method described in Example 20.

In an embodiment of the present invention, the protein content of the product is at least about 60 wt % (N×6.25) d.b. and the phytic acid content of the product is less than about 1.5 wt %.

In an embodiment of the present invention, the protein content of the product is at least about 60 wt % (N×6.25) d.b. and the protein solubility at 1% protein w/v in water at a pH of about 2 to about 4 is greater than about 90%. In an embodiment of the present invention, the protein solubility is determined by the protein method described in Example 3.

In an embodiment of the present invention, the protein content of the product is at least about 60 wt % (N×6.25) d.b. and the L* reading for a solution prepared by dissolving sufficient protein powder to supply 0.48 g of protein in 15 ml of water is greater than about 96.50. In an embodiment of the present invention, the b* reading for a solution prepared by dissolving sufficient protein powder to supply 0.48 g of protein in 15 ml of water is less than about 8. In an embodiment of the present invention, the b* reading for the dry protein powder is less than about 8.

In an embodiment of the present invention, the second retentate containing the larger, more astringent protein species may optionally be adjusted in pH to about 6.0 to about 8.0 and then optionally dried. The pH adjusted product is intended typically for use in neutral applications, such as processed meat products, baked goods, nutrition bars and dairy analogue or alternative products.

In accordance with another aspect of the present invention, there is provided a soy protein product having a molecular weight profile, which is:

about 25 to about 100% greater than about 100,000 Da;

about 0 to about 50% from about 15,000 to about 100,000 Da;

about 0 to about 18% from about 5,000 to about 15,000 Da; and

about 0 to about 42% from about 1,000 to about 5,000 Da.

In an embodiment of the present invention, the molecular weight profile is:

about 35 to about 95% greater than about 100,000 Da;

about 2 to about 30% from about 15,000 to about 100,000 Da;

about 0 to about 7% from about 5,000 to about 15,000 Da; and

about 0 to about 35% from about 1,000 to about 5,000 Da.

In accordance with another aspect of the present invention, there is provided a soy protein product having a molecular weight profile, which is:

about 32 to about 100% greater than about 100,000 Da;

about 0 to about 34% from about 15,000 to about 100,000 Da;

about 0 to about 12% from about 5,000 to about 15,000 Da; and

about 0 to about 40% from about 1,000 to about 5,000 Da.

In an embodiment of the present invention, the molecular weight profile is:

about 37 to about 95% greater than about 100,000 Da;

about 0 to about 29% from about 15,000 to about 100,000 Da;

about 0 to about 7% from about 5,000 to about 15,000 Da; and

about 1 to about 35% from about 1,000 to about 5,000 Da.

In an embodiment of the present invention, the molecular weight profile is determined by size exclusion chromatography at a pH of about 3.5. In another embodiment of the present invention, the molecular weight profile is determined by the method described in Example 19.

In an embodiment of the present invention, the protein content of the product is at least about 60 wt % (N×6.25) d.b. and the protein solubility at 1% protein w/v in water at a pH of about 2 to about 3 is less than about 60%. In an embodiment of the present invention, the protein solubility at 1% protein w/v in water at a pH of about 4 is between about 5 and about 30%. In an embodiment of the present invention, the protein solubility is determined by the protein method described in Example 14.

In an embodiment of the present invention, the protein content of the product is at least about 60 wt % (N×6.25) d.b. and the b* reading for the dry protein powder is greater than about 8.2.

In an embodiment of the present invention, the protein content of the product is at least about 60 wt % (N×6.25) d.b. and the phytic acid content of the product is less than about 1 wt %.

In accordance with another aspect of the present invention, there is provided a soy protein product having a molecular weight profile, which is:

about 10 to about 38% greater than about 100,000 Da;

about 13 to about 33% from about 15,000 to about 100,000 Da;

about 0 to about 12% from about 5,000 to about 15,000 Da; and

about 36 to about 62% from about 1,000 to about 5,000 Da.

In an embodiment of the present invention, the molecular weight profile is:

about 15 to about 33% greater than about 100,000 Da;

about 18 to about 28% from about 15,000 to about 100,000 Da;

about 5 to about 8% from about 5,000 to about 15,000 Da; and

about 41 to about 57% from about 1,000 to about 5,000 Da.

In an embodiment of the present invention, the molecular weight profile is determined by size exclusion chromatography at a pH of about 6. In another embodiment of the present invention, the molecular weight profile is determined by the method described in Example 20.

In an embodiment of the present invention, the protein content of the product is at least about 60 wt % (N×6.25) d.b. and the protein solubility at 1% protein w/v in water at a pH of about 2 to about 3 is less than about 60%. In an embodiment of the present invention, the protein solubility at 1% protein w/v in water at a pH of about 4 is less than about 30%. In an embodiment of the present invention, the protein solubility at 1% protein w/v in water at a pH of about 5 is less than about 10%. In an embodiment of the present invention, the protein solubility at 1% protein w/v in water at a pH of about 6 is less than about 15%. In an embodiment of the present invention, the protein solubility at 1% protein w/v in water at a pH of about 7 is less than about 25%. In an embodiment of the present invention, the protein solubility is determined by the protein method described in Example 14.

The reduced astringency soy protein products of the present invention, produced according to the processes of the present invention disclosed herein, are particularly suitable for use in protein fortification of acid media. However, the reduced astringency soy protein products of the present invention as well as the co-products of their production, containing the higher astringency proteins, may also be used in a wide variety of conventional applications of protein products, including but not limited to protein fortification of processed foods and beverages and as functional ingredients in foods and beverages. The soy protein products of the present invention may also be used in dairy analogue or dairy alternative products, including products that are dairy/plant ingredient blends. The soy protein products may also be used in nutritional supplements. Other uses of the soy protein products of the present invention would be understood by persons skilled in the art and include, but are not limited to, use in pet foods, animal feed and in industrial and cosmetic applications and in personal care products.

### General Description of Invention

The initial step of the process of the present invention of providing the soy protein products of the present invention involves solubilizing soy protein from a soy protein source. The soy protein source may be soybeans or any soy product or by-product derived from the processing of soybeans, including but not limited to soy meal, soy flakes, soy grits and soy flour. The soy protein source may be used in the full fat form, partially defatted form or fully defatted form. Where the soy protein source contains an appreciable amount of fat, an oil removal step generally is required during the process. The soy protein recovered from the soy protein source may be the protein naturally occurring in soybean or the proteinaceous material may be a protein modified by genetic manipulation but possessing characteristic hydrophobic and polar properties of the natural protein.

Protein solubilization from the soy protein source material is effected most conveniently using calcium chloride solution, although solutions of other calcium salts may be used. In addition, other alkaline earth metal compounds may be used, such as, for example, but not limited to, magnesium salts. Further, extraction of the soy protein from the soy protein source may be effected using calcium salt solution in combination with another salt solution, such as, for example, but not limited to, sodium chloride. Alternatively, extraction of the soy protein from the soy protein source may be effected using water or other salt solution, such as, for example, but not limited to, sodium chloride, with calcium salt subsequently being added to the aqueous soy protein solution produced in the extraction step. Precipitate formed upon addition of the calcium salt is removed prior to subsequent processing.

As the concentration of the calcium salt solution increases, the degree of solubilization of protein from the soy protein source initially increases until a maximum value is achieved. Any subsequent increase in salt concentration does not increase the total protein solubilized. The concentration of calcium salt solution which causes maximum protein solubilization varies depending on the salt concerned. It is usually preferred to utilize a concentration value less than about 1.0 M, and more preferably a value of about 0.10 to about 0.15 M.

In a batch process, the salt solubilization of the protein is effected at a temperature of from about 1° to about 100° C., preferably about 15° C. to about 65° C., more preferably about 50° to about 60° C., preferably accompanied by agitation to decrease the solubilization time, which is usually about 1 to about 60 minutes. It is preferred to effect the solubilization to extract substantially as much protein from the soy protein source as is practical, so as to provide an overall high product yield.

In a continuous process, the extraction of the soy protein from the soy protein source is carried out in any manner consistent with effecting a continuous extraction of protein from the soy protein source. In one embodiment, the soy protein source is continuously mixed with the calcium salt solution and the mixture is conveyed through a pipe or conduit having a length and at a flow rate for a residence time sufficient to effect the desired extraction in accordance with the parameters described herein. In such a continuous procedure, the salt solubilization step is effected in a time of about 1 minute to about 60 minutes, preferably to effect solubilization to extract substantially as much protein from the soy protein source as is practical. The solubilization in the continuous procedure is effected at temperatures between about 1° and about 100° C., preferably between about 15° C. and about 65° C., more preferably between about 50° and about 60° C.

The extraction is generally conducted at a pH of about 4.5 to about 11, preferably about 5 to about 7. The pH of the extraction system (soy protein source and calcium salt solution) may be adjusted to any desired value within the range of about 4.5 to about 11 for use in the extraction step by the use of any conventional food grade acid, usually hydrochloric acid or phosphoric acid or mixtures thereof, preferably hydrochloric acid, as required, or food grade alkali, usually sodium hydroxide or potassium hydroxide or mixtures thereof, preferably sodium hydroxide, as required.

The concentration of soy protein source in the calcium salt solution during the solubilization step may vary widely. Typical concentration values are about 5 to about 15% w/v.

The protein extraction step with the aqueous calcium salt solution has the additional effect of solubilizing fats which may be present in the soy protein source, which then results in the fats being present in the aqueous phase.

The protein solution resulting from the extraction step generally has a protein concentration of about 5 to about 50 g/L, preferably about 10 to about 50 g/L.

The aqueous calcium salt solution used for extraction may contain an antioxidant. The antioxidant may be any conventional antioxidant, such as, for example, but not limited to, sodium sulfite or ascorbic acid or mixtures thereof. The quantity of antioxidant employed may vary from about 0.01 to about 1 wt % of the solution, preferably about 0.05 wt %. The antioxidant serves to inhibit oxidation of any phenolics in the protein solution.

The aqueous protein solution resulting from the extraction step then may be separated from the residual soy protein source, in any conventional manner, such as by employing a decanter centrifuge or any suitable sieve, followed by disc centrifugation and/or filtration, to remove residual soy protein source material. The separation step is typically conducted at the same temperature as the protein solubilisation step, but may be conducted at any temperature within the range of about 1° to about 100° C., preferably about 15° to about 65° C., more preferably about 50° to about 60° C. The separated residual soy protein source may be dried for disposal or further processed to recover residual protein. The separated residual soy protein source may be re-extracted with fresh calcium salt solution and the protein solution yielded upon clarification combined with the initial protein solution for further processing as described below. A counter-current extraction procedure may also be utilized. Alternatively, the separated residual soy protein source may be processed by any other conventional procedure to recover residual protein.

The aqueous soy protein solution may be treated with an anti-foamer, such as any conventionally suitable food-grade, non-silicone based anti-foamer, to reduce the volume of foam formed upon further processing. The quantity of anti-foamer employed is generally greater than about 0.0003% w/v. Alternatively, the anti-foamer in the quantity described may be added in the extraction steps.

Where the soy protein source contains significant quantities of fat, as described in U.S. Pat. Nos. 5,844,086 and 6,005,076, assigned to the assignee hereof and the disclosures of which are incorporated herein by reference, then the defatting steps described therein may be effected on the separated aqueous protein solution. Alternatively, defatting of the separated aqueous soy protein solution may be achieved by any other conventional procedure.

The aqueous soy protein solution may be treated with an adsorbent, such as powdered activated carbon or granulated activated carbon, to remove colour and/or odour compounds. Such adsorbent treatment may be carried out under any conventional conditions, generally at the ambient temperature of the separated aqueous protein solution. For powdered activated carbon, an amount of about 0.025% to about 5% w/v, preferably about 0.05% to about 2% w/v, may be employed. The adsorbing agent may be removed from the soy protein solution by any conventional means, such as by filtration.

The resulting aqueous soy protein solution may be diluted generally with about 0.1 to about 10 volumes, preferably about 0.5 to about 2 volumes of aqueous diluent, in order to decrease the conductivity of the aqueous soy protein solution to a value of generally below about 105 mS, preferably about 4 to about 21 mS. Such dilution is usually effected using water, although dilute salt solution, such as sodium chloride or calcium chloride, having a conductivity up to about 3 mS, may be used.

The diluent with which the soy protein solution is mixed generally has the same temperature as the soy protein solution, but the diluent may have a temperature of about 1° to about 100° C., preferably about 15° to about 65° C., more preferably about 50° to about 60° C.

The optionally diluted soy protein solution then is adjusted in pH to a value of about 1.5 to about 4.4, preferably about 2 to about 4, by the addition of any conventionally suitable food grade acid, such as, for example, but not limited to, hydrochloric acid or phosphoric acid or mixtures thereof, preferably hydrochloric acid, to result in a clear acidified aqueous soy protein solution. The clear acidified aqueous soy protein solution has a conductivity of generally below about 110 mS for a diluted soy protein solution, or generally below about 115 mS for an undiluted soy protein solution, in both cases preferably about 4 to about 26 mS.

As described in co-pending U.S. patent application Ser. No. 13/474,788 filed May 18, 2012 (“S704”) (published as US Patent Application Publication No. 20120295008), assigned to the assignee hereof and the disclosure of which is incorporated herein by reference, the optional dilution and acidification steps may be effected prior to separation of the soy protein solution from the residual soy protein source material.

The clear acidified aqueous soy protein solution may be subjected to a heat treatment to inactivate heat labile anti-nutritional factors, such as trypsin inhibitors, present in such solution as a result of extraction from the soy protein source material during the extraction step. Such a heating step also provides the additional benefit of reducing the microbial load. Generally, the protein solution is heated to a temperature of about 70° to about 160° C. for about 10 seconds to about 60 minutes, preferably about 80° to about 120° C. for about 10 seconds to about 5 minutes, more preferably about 85° to about 95° C. for about 30 seconds to about 5 minutes. The heat treated acidified soy protein solution then may be cooled for further processing as described below, to a temperature of about 2° to about 65° C., preferably about 50° C. to about 60° C.

The optionally diluted, acidified and optionally heat treated protein solution may optionally be polished by any conventional means, such as by filtering, to remove any residual particulates.

In accordance with an aspect of the present invention, the acidified aqueous soy protein solution is subjected to concentration and/or diafiltration steps in such a way as to separate lower molecular weight, less astringent proteins from higher molecular weight, more astringent proteins. The molecular weight cut-off of the concentration and diafiltration membranes are chosen to permit the smaller, less astringent proteins to pass to the permeate with the contaminants. Such concentration and/or diafiltration steps may be effected in any conventional manner consistent with batch or continuous operation, such as by employing any conventional selective membrane technique, such as microfiltration or ultrafiltration, using membranes, such as hollow-fibre membranes or spiral-wound membranes, with a suitable molecular weight cut-off, such as about 0.02 to about 0.1 μm, preferably about 0.08 to about 0.1 μm for microfiltration and about 5,000 to about 1,000,000 daltons, preferably about 50,000 to about 1,000,000 daltons for ultrafiltration, having regard to differing membrane materials and configurations, and, for continuous operation, dimensioned to permit the desired degree of concentration as the aqueous protein solution passes through the membranes. In the concentration step the acidified protein solution is concentrated to a protein concentration of about 50 to about 300 g/L, preferably about 100 to about 200 g/L. The acidified protein solution may also be partially concentrated to a protein concentration of less than about 50 g/L.

As is well known, ultrafiltration and similar selective membrane techniques permit low molecular weight species to pass therethrough while preventing higher molecular weight species from so doing. The low molecular weight species include not only the ionic species of the salt but also low molecular weight materials extracted from the source material, such as carbohydrates, pigments and low molecular weight proteins including the less astringent proteins and the anti-nutritional trypsin inhibitors.

The concentrated protein solution may be diafiltered with water or dilute salt solution. The diafiltration solution may be at its natural pH or at a pH equal to that of the protein solution being diafiltered or any pH value in between. Such diafiltration may be effected using from about 1 to about 40 volumes of diafiltration solution, preferably about 2 to about 25 volumes of diafiltration solution. The concentration and optional diafiltration steps may be effected at any conventional temperature, generally about 2° to about 65° C., preferably about 50° to about 60° C. The smaller less astringent proteins are captured in the permeate of the membrane processes along with other small molecule contaminants.

The diafiltration step may alternatively be applied to the acidified aqueous protein solution prior to concentration or to partially concentrated acidified aqueous protein solution. Diafiltration may also be applied at multiple points during the concentration process. When diafiltration is applied prior to concentration or to partially concentrated solution, the resulting diafiltered solution may then be fully concentrated. Viscosity reduction achieved by diafiltering multiple times as the protein solution is concentrated may allow a higher final, fully concentrated protein concentration to be achieved.

An antioxidant may be present in the diafiltration medium during at least part of the diafiltration step. The antioxidant may be any conventional antioxidant, such as, for example, but not limited to, sodium sulfite or ascorbic acid or mixtures thereof. The quantity of antioxidant employed in the diafiltration medium depends on the materials employed and may vary from about 0.01 to about 1 wt %, preferably about 0.05 wt %. The antioxidant serves to inhibit the oxidation of any phenolics present in the concentrated soy protein solution.

The less astringent proteins are then separated from the contaminants by subsequent concentration and/or diafiltration of the protein solution (first permeate) by membrane processing, such as ultrafiltration using membranes having a lower molecular weight cut-off such as about 1,000 to about 100,000 daltons, preferably about 1,000 to about 10,000 daltons, operated as described above. The less astringent proteins are collected in the retentate of this second membrane step (second retentate), while the contaminants pass through the membrane to the (second) permeate. In the concentration step, the first permeate is concentrated to a protein concentration of about 10 to about 300 g/L, preferably about 100 to about 200 g/L. The protein solution (first permeate) may also be partially concentrated to a protein concentration of less than about 10 g/L.

The first permeate may be diafiltered before or after partial or complete concentration thereof according to the conditions described above. When diafiltration is applied prior to concentration or to partially concentrated solution, the resulting diafiltered solution may then be fully concentrated.

Additional products may be obtained from the retentate of the membrane fractionation process (first retentate), which contains the more astringent proteins. This protein solution may be optionally dried by any conventional means, with or without adjustment of the pH of the protein solution to about 6 to about 8 using any conventionally suitable food grade alkali, such as, for example, but not limited to sodium hydroxide or potassium hydroxide or mixtures thereof, preferably sodium hydroxide.

The concentration and diafiltration steps employed in the purification of the aqueous solution of less astringent proteins derived from the first permeate in the membrane fractionation procedure may be effected herein in such a manner that the reduced astringency soy protein product recovered contains less than about 90 wt % protein (N×6.25) d.b., such as at least about 60 wt % protein (N×6.25) d.b. By partially concentrating and/or partially diafiltering the first permeate, it is possible to only partially remove contaminants. This protein solution (second retentate) may then be dried to provide a soy protein product with lower levels of purity. The soy protein products of the present invention are highly soluble and able to produce less astringent protein solutions, preferably clear, less astringent protein solutions, under acidic conditions.

As alluded to earlier, soy contains anti-nutritional trypsin inhibitors. The level of trypsin inhibitor activity in the final soy protein products can be controlled by the manipulation of various process variables.

Heat treatment of the acidified aqueous soy protein solution may be used to inactivate heat-labile trypsin inhibitors. The partially concentrated or fully concentrated soy protein solution may also be heat treated to inactivate heat labile trypsin inhibitors. Such a heat treatment may also be applied to the solution of less astringent, lower molecular weight proteins arising from the membrane separation method (first permeate), before or after partial or complete concentration. When the heat treatment is applied to a solution that is not already fully concentrated, the resulting heat treated solution may then be additionally concentrated.

Membrane processing the soy protein solutions at a lower pH, such as about 1.5 to about 3, preferably 1.5 to 3, may reduce the trypsin inhibitor activity relative to processing the solutions at higher pH, such as about 3 to about 4.4, preferably 3 to 4.4. When the first permeate is concentrated and diafiltered at the low end of the pH range, it may be desired to raise the pH of the retentate prior to drying. The pH of the concentrated and diafiltered protein solution may be raised to the desired value, for example pH of about 3, by the addition of any conventional food grade alkali, such as, for example, but not limited to, sodium hydroxide or potassium hydroxide or mixtures thereof, preferably sodium hydroxide.

Further, a reduction in trypsin inhibitor activity may be achieved by exposing soy materials to reducing agents that disrupt or rearrange the disulfide bonds of the inhibitors. Suitable reducing agents include for example, but not limited to, sodium sulfite, cysteine and N-acetylcysteine and combinations thereof.

The addition of such reducing agents may be effected at various stages of the overall process. The reducing agent may be added with the soy protein source material in the extraction step, may be added to the clarified aqueous soy protein solution following removal of residual soy protein source material, may be added to the second retentate before drying or may be dry blended with the dried soy protein product. The addition of the reducing agent may be combined with the heat treatment step and membrane processing steps, as described above.

If it is desired to retain active trypsin inhibitors in the protein products, this can be achieved by eliminating or reducing the intensity of the heat treatment step, not utilizing reducing agents, and/or operating the concentration and diafiltration steps at the higher end of the pH range, such as about 3 to about 4.4, preferably 3 to 4.4.

The concentrated and/or diafiltered protein solutions described above may be subject to a further defatting operation, if required, as described in U.S. Pat. Nos. 5,844,086 and 6,005,076. Alternatively, defatting of the concentrated and/or diafiltered protein solutions may be achieved by any other conventional procedure.

The concentrated and/or diafiltered aqueous protein solutions described above may be treated with an adsorbent, such as powdered activated carbon or granulated activated carbon, to remove colour and/or odour compounds. Such adsorbent treatment may be carried out under any conventional conditions, generally at the ambient temperature of the concentrated protein solution. For powdered activated carbon, an amount of about 0.025% to about 5% w/v, preferably about 0.05% to about 2% w/v, may be employed. The adsorbent may be removed from the soy protein solution by any conventional means, such as by filtration.

The first and second retentate solutions described above may be dried by any conventional technique, such as spray drying or freeze drying. A pasteurization step may be effected on the first and second retentate solutions prior to drying. Such pasteurization may be effected under any desired pasteurization conditions. Generally, retentate solutions are heated to a temperature of about 55° to about 75° C. for about 15 seconds to about 60 minutes. The pasteurized soy protein solutions then may be cooled for drying, preferably to a temperature of about 25° to about 40° C.

Each of the soy protein products of the present invention obtained by the procedures of the present invention described above has a protein content of at least about 60 wt % (N×6.25) d.b. Preferably, the soy protein products of the present invention are isolates with a protein content in excess of about 90 wt % (N×6.25) d.b., preferably at least about 100 wt %, (N×6.25) d.b.

The less astringent soy protein products of the present invention produced by the procedures of the present invention disclosed herein are soluble in an acidic aqueous environment, making the products ideal for incorporation into beverages to provide protein fortification thereto. Such beverages have a wide range of acidic pH values, ranging from about 2.5 to about 5. The soy protein products of the present invention may be added to such beverages in any convenient quantity to provide protein fortification to such beverages, for example, at least about 5 g of the soy protein per serving. The added soy protein product dissolves in the beverage and the haze level of the beverage is not increased by thermal processing. The soy protein product may be blended with dried beverage prior to reconstitution of the beverage by dissolution in water. In some cases, modification to the normal formulation of the beverages to tolerate the composition of the present invention may be necessary where components present in the beverage may adversely affect the ability of the composition of the present invention to remain dissolved in the beverage.

### EXAMPLES

### Example 1

This Example illustrates production of the reduced astringency soy protein product of the present invention with membrane processing utilized to separate the less astringent proteins from the more astringent proteins.

‘a’ kg of soy white flake was added to ‘b’ L of ‘c’ M CaCl₂ solution and the mixture stirred for 30 minutes at about 60° C. Insoluble material was removed and the sample clarified by centrifugation, yielding ‘d’ L of protein extract solution having a protein concentration of ‘e’ wt %. ‘f’ L of protein extract solution was combined with ‘g’ L of reverse osmosis (RO) purified water and the pH of the sample lowered to ‘h’ with HCl solution (HCl diluted with an equal volume of water). ‘i’ L of acidified protein solution was reduced in volume to ‘j’ L using a polyvinyldifluoridene microfiltration membrane having a pore size of 0.08 μm and operated at about ‘k’ ° C. The microfiltration retentate was then diafiltered with ‘l’ L of RO purified water at about ‘m’ ° C. and then the diafiltered retentate further reduced to ‘n’ at about ‘o’ ° C. ‘p’ L of microfiltration/diafiltration permeate having a protein concentration of ‘q’ wt % was reduced in volume to ‘r’ L using a PES ultrafiltration membrane having a pore size of ‘s’ daltons operated at a temperature of about ‘t’ ° C. The ‘u’ concentrated protein solution was then diafiltered with ‘v’ L of RO purified water at about ‘w’ ° C. to provide ‘x’ of ‘y’ concentrated and diafiltered protein solution having a protein content of ‘z’ wt %. This solution was then further concentrated at a temperature of about ‘aa’ to provide ‘ab’ kg of solution having a protein content of ‘ac’ wt %. This represented a yield of ‘ad’ % of the protein in the protein extract solution. ‘ae’ kg of the ‘af’ concentrated and diafiltered protein solution was spray dried to yield a protein product, having a protein content of ‘ag’ % (N×6.25) d.b., termed ‘1h’ S706.

The ‘n’ of concentrated and diafiltered microfiltration retentate collected, having a protein content of ‘ai’ wt % represented a yield of ‘aj’ % of the protein in the protein extract solution resulting from the clarification step after calcium chloride addition. ‘n’ of concentrated and diafiltered microfiltration retentate was diluted with ‘ak’ L of water, adjusted to pH ‘al’, ‘am’ and then at least an aliquot of the material spray dried to form a protein product having a protein content of ‘an’ % (N×6.25) d.b., termed ‘ah’ S706B.

The parameters ‘a’ to ‘an’ are set forth in the following Table 1.

TABLE 1
Parameters for the production of S706 and S706B

| ah | S024-J01-13A | S024-J21-13A | S024-F11-14A | S024-F12-14A |
|---|---|---|---|---|
| a | 35 | 35 | 60 | 60 |
| b | 350 | 350 | 600 | 600 |
| c | 0.08 | 0.08 | 0.09 | 0.09 |
| d | 285 | 275 | 483 | 480 |
| e | 2.66 | 2.58 | 2.75 | 2.79 |
| f | 285 | 275 | 483 | 480 |
| g | 183 | 185 | 300 | 306 |
| h | 3.24 | 2.70 | 3.29 | 3.22 |
| i | 477 | 480 | 790 | 790 |
| j | 122 | 111 | 172 | 175 |
| k | 52 | 50 | 54 | 50 |
| l | 183 | 222 | 344 | 350 |
| m | 49 | 50 | 55 | 49 |
| n | 63.04 kg | 52.64 kg | 77 L | 85 L |
| o | 53 | 50 | 53 | 50 |
| p | 595 | 649 | 1046 | 1062 |
| q | 0.28 | about 0.23 | about 0.21 | about 0.24 |
| r | 20 | 32.5 | 70 | 58 |
| s | 100,000 | 100,000 | 1,000 | 1,000 |
| t | 53 | 48 | 44 | 44 |
| u | Partially | Not applicable | Not applicable | Not applicable |
| v | 100 | 162.5 | 350 | 290 |
| w | 55 | 51 | 49 | 49 |
| x | 17.9 kg | 32.5 L | 70 L | 58 L |
| y | Partially | Not applicable | Not applicable | N/A |
| z | 0.97 | 0.77 | 1.91 | 2.33 |
| aa | N/A | Not recorded | 51 | 51 |
| ab | N/A | 18.96 | 34.08 | 34.23 |
| ac | N/A | 2.41 | 3.76 | 3.80 |
| ad | 2.2 | 6.5 | 9.6 | 9.7 |
| ae | 17.9 | 18.96 | 34.08 | 34.23 |
| af | partially | Not applicable | Not applicable | Not applicable |
| ag | 99.67 | 102.62 | 101.14 | 101.51 |
| ai | 8.91 | 10.08 | 11.19 | 10.81 |
| aj | 74.1 | 74.8 | 64.9 | 68.6 |
| ak | Not applicable | Not applicable | 70 | 67 |
| al | 7.11 | about 7 | Not applicable | 7.74 |
| am | Not applicable | diluted with | Not applicable | Not applicable |
| | | 26 L of water | | |
| an | 96.10 | 97.61 | 100.51 | 95.82 |

### Example 2

This Example contains an evaluation of the dry colour and colour in solution of the reduced astringency soy protein products produced by the methods of Example 1.

The colour of the dry powders was assessed using a HunterLab ColorQuest XE instrument in reflectance mode. The colour values are set forth in the following Table 2:

TABLE 2
HunterLab scores for dry protein products

| Sample | L* | a* | b* |
|---|---|---|---|
| S024-J01-13A S706 | 88.43 | 0.37 | 6.05 |
| S024-J21-13A S706 | 88.55 | 0.32 | 6.16 |
| S024-F11-14A S706 | 88.49 | 0.29 | 7.08 |
| S024-F12-14A S706 | 88.36 | 0.31 | 7.21 |

As may be seen from Table 2, the reduced astringency soy protein products were light in colour.

Solutions of the reduced astringency soy protein products were prepared by dissolving sufficient protein powder to supply 0.48 g of protein in 15 ml of RO purified water. The pH of the solutions was measured with a pH meter and the colour and clarity assessed using a HunterLab ColorQuest XE instrument operated in transmission mode. The results are shown in the following Table 3.

TABLE 3
pH and HunterLab scores for solutions of
reduced astringency soy protein products

| sample | pH | L* | a* | b* | haze |
|---|---|---|---|---|---|
| S024-J01-13A S706 | 3.87 | 96.91 | −0.47 | 7.55 | 6.8 |
| S024-J21-13A S706 | 3.80 | 97.13 | −0.49 | 7.42 | 3.6 |
| S024-F11-14A S706 | 3.82 | 97.27 | −0.55 | 7.44 | 3.0 |
| S024-F12-14A S706 | 3.85 | 97.44 | −0.59 | 7.17 | 2.0 |

As may be seen from the results in Table 3, the solutions of the reduced astringency soy protein products were light in colour and low in haze.

### Example 3

This Example contains an evaluation of the solubility in water of the reduced astringency soy protein products produced by the methods of Example 1. Solubility was tested based on protein solubility (termed protein method, a modified version of the procedure of Morr et al., J. Food Sci. 50:1715-1718) and total product solubility (termed pellet method).

Sufficient protein powder to supply 0.5 g of protein was weighed into a beaker and then a small amount of RO purified water was added and the mixture stirred until a smooth paste formed. Additional water was then added to bring the volume to approximately 45 ml. The contents of the beaker were then slowly stirred for 60 minutes using a magnetic stirrer. The pH was determined immediately after dispersing the protein and was adjusted to the appropriate level (2, 3, 4, 5, 6 or 7) with diluted NaOH or HCl. A sample was also prepared at natural pH. For the pH adjusted samples, the pH was measured and corrected periodically during the 60 minutes stirring. After the 60 minutes of stirring, the samples were made up to 50 ml total volume with RO purified water, yielding a 1% w/v protein dispersion. The protein content of the dispersions was determined by combustion analysis using a Nitrogen Determinator (Leco Corporation, St. Joseph, Mich.). Aliquots (20 ml) of the dispersions were then transferred to pre-weighed centrifuge tubes that had been dried overnight in a 100° C. oven then cooled in a desiccator and the tubes capped. The samples were centrifuged at 7,800 g for 10 minutes, which sedimented insoluble material and yielded a supernatant. The protein content of the supernatant was measured by combustion analysis and then the supernatant and the tube lids were discarded and the pellet material dried overnight in an oven set at 100° C. The next morning the tubes were transferred to a desiccator and allowed to cool. The weight of dry pellet material was recorded. The dry weight of the initial protein powder was calculated by multiplying the weight of powder used by a factor of ((100−moisture content of the powder (%))/100). Solubility of the product was then calculated two different ways:

Solubility(protein method)(%)=(% protein in supernatant/% protein in initial dispersion)×100 1)

Solubility(pellet method)(%)=(1−(weight dry insoluble pellet material/((weight of 20 ml of dispersion/weight of 50 ml of dispersion)×initial weight dry protein powder)))×100 2)

Values calculated as greater than 100% were reported as 100%.

The solubility results obtained are set forth in the following Tables 4 and 5:

TABLE 4
Solubility of products at different
pH values based on protein method

| | | Solubility (protein method) (%) | | | | | |
|---|---|---|---|---|---|---|---|
| Batch | Product | pH 2 | pH 3 | pH 4 | pH 5 | pH 6 | pH 7 |
| S024-J21-13A | S706 | 100 | 100 | 100 | 26.7 | 31.7 | 92.5 |
| S024-F12-14A | S706 | 100 | 98.1 | 98.1 | 37.5 | 36.4 | 99.0 |

TABLE 5
Solubility of products at different
pH values based on pellet method

| | | Solubility (pellet method) (%) | | | | | |
|---|---|---|---|---|---|---|---|
| Batch | Product | pH 2 | pH 3 | pH 4 | pH 5 | pH 6 | pH 7 |
| S024-J21-13A | S706 | 98.4 | 98.6 | 98.5 | 40.5 | 36.7 | 91.7 |
| S024-F12-14A | S706 | 99.0 | 99.9 | NA | 47.0 | 60.9 | 98.0 |

NA = Not Available

As can be seen from the results presented in Tables 4 and 5, the reduced astringency soy protein products were highly soluble in the pH range 2-4 and also had very good solubility at pH 7.

### Example 4

This Example contains an evaluation of the clarity in water of the reduced astringency soy protein products produced by the methods of Example 1.

The clarity of the 1% w/v protein solutions prepared as described in Example 3 was assessed by measuring the absorbance at 600 nm (water blank), with a lower absorbance score indicating greater clarity. Analysis of the samples on a HunterLab ColorQuest XE instrument in transmission mode also provided a percentage haze reading, another measure of clarity.

The clarity results are set forth in the following Tables 6 and 7:

TABLE 6
Clarity of protein solutions at different
pH values as assessed by A600

| | | A600 | | | | | |
|---|---|---|---|---|---|---|---|
| Batch | Product | pH 2 | pH 3 | pH 4 | pH 5 | pH 6 | pH 7 |
| S024-J21-13A | S706 | 0.010 | 0.011 | 0.019 | 2.507 | 2.433 | 0.540 |
| S024-F12-14A | S706 | 0.007 | 0.008 | 0.015 | 1.937 | 2.242 | 0.139 |

TABLE 7
Clarity of protein solutions at different pH
values as assessed by HunterLab haze analysis

| | | HunterLab haze reading (%) | | | | | |
|---|---|---|---|---|---|---|---|
| Batch | Product | pH 2 | pH 3 | pH 4 | pH 5 | pH 6 | pH 7 |
| S024-J21-13A | S706 | 2.0 | 2.0 | 4.3 | 98.2 | 97.4 | 77.4 |
| S024-F12-14A | S706 | 2.5 | 1.0 | 4.1 | NA | 99.7 | 21.8 |

NA = Not Available

As can be seen from the results of Tables 6 and 7, the reduced astringency soy protein products provided solutions that were low in haze at pH 2-4.

### Example 5

This Example contains an evaluation of the solubility in a soft drink (Sprite) and sports drink (Orange Gatorade) of the reduced astringency soy protein products produced by the method of Example 1. The solubility was determined with the protein added to the beverages with no pH correction and again with the pH of the protein fortified beverages adjusted to the level of the original beverages.

When the solubility was assessed with no pH correction, a sufficient amount of protein powder to supply 1 g of protein was weighed into a beaker and then a small amount of beverage was added and the mixture stirred until a smooth paste formed. Additional beverage was then added to bring the volume to 50 ml, and then the solutions were stirred slowly on a magnetic stirrer for 60 minutes to yield a 2% protein w/v dispersion. The protein content of the samples was determined by combustion analysis then an aliquot of the protein containing beverages was centrifuged at 7,800 g for 10 minutes and the protein content of the supernatant measured.

Solubility(%)=(% protein in supernatant/% protein in initial dispersion)×100.

Values calculated as greater than 100% were reported as 100%.

When the solubility was assessed with pH correction, the pH of the soft drink (Sprite) and sports drink (Orange Gatorade) without protein was measured. A sufficient amount of protein powder to supply 1 g of protein was weighed into a beaker and then a small amount of beverage was added and the mixture stirred until a smooth paste formed. Additional beverage was added to bring the volume to approximately 45 ml, and then the solutions were stirred slowly on a magnetic stirrer for 60 minutes. The pH of the protein containing beverages was determined immediately after dispersing the protein and was adjusted to the original no-protein pH with HCl or NaOH as necessary. The pH was measured and corrected periodically during the 60 minutes stirring. After the 60 minutes of stirring, the total volume of each solution was brought to 50 ml with additional beverage, yielding a 2% protein w/v dispersion. The protein content of the samples was determined by combustion analysis then an aliquot of the protein containing beverages was centrifuged at 7,800 g for 10 minutes and the protein content of the supernatant measured.

Solubility (%)=(% protein in supernatant/% protein in initial dispersion)×100

Values calculated as greater than 100% were reported as 100%.

The results obtained are set forth in the following Table 8:

TABLE 8
Solubility of reduced astringency soy protein
products in Sprite and Orange Gatorade

| | | no pH correction | | pH correction | |
|---|---|---|---|---|---|
| | | | Solubility | | Solubility |
| | | Solubility | (%) in | Solubility | (%) in |
| | | (%) in | Orange | (%) in | Orange |
| Batch | Product | Sprite | Gatorade | Sprite | Gatorade |
| S024-J21-13A | S706 | 92.6 | 100 | 100 | 100 |
| S024-F12-14A | S706 | 100 | 100 | 100 | 100 |

As can be seen from the results of Table 8, the reduced astringency soy protein products were highly soluble in the Sprite and the Orange Gatorade.

### Example 6

This Example contains an evaluation of the clarity in a soft drink and sports drink of the reduced astringency soy protein products produced by the method of Example 1.

The clarity of the 2% w/v protein dispersions prepared in soft drink (Sprite) and sports drink (Orange Gatorade) in Example 5 were assessed using the HunterLab haze method described in Example 4.

The results obtained are set forth in the following Table 9:

TABLE 9
HunterLab haze readings for reduced astringency soy
protein products in Sprite and Orange Gatorade

| | | no pH correction | | pH correction | |
|---|---|---|---|---|---|
| | | | Haze | | Haze |
| | | Haze | (%) in | Haze | (%) in |
| | | (%) in | Orange | (%) in | Orange |
| Batch | Product | Sprite | Gatorade | Sprite | Gatorade |
| no protein | | 0.0 | 77.2 | 0.0 | 77.2 |
| S024-J21-13A | S706 | 4.6 | 63.6 | 4.3 | 70.1 |
| S024-F12-14A | S706 | 2.2 | 63.0 | 2.6 | 69.3 |

As can be seen from the results of Table 9, the addition of the reduced astringency soy protein products to the soft drink and sports drink added little or no haze.

### Example 7

This Example contains an evaluation of the heat stability in water of the reduced astringency soy protein products produced by the methods of Example 1.

2% w/v protein solutions of the protein products were prepared in RO purified water with the pH of the solutions adjusted to about 3.0 with HCl solution. The clarity of the solutions was assessed by haze measurement with the HunterLab ColorQuest XE instrument operated in transmission mode. The solutions were then heated to 95° C., held at this temperature for 30 seconds and then immediately cooled to room temperature in an ice bath. The clarity of the heat treated solutions was then measured again.

The clarity of the protein solutions before and after heating is set forth in the following Table 10:

TABLE 10
Effect of heat treatment on clarity of 2% w/v protein
solutions of reduced astringency soy protein products

| | | haze before heat | haze after heat |
|---|---|---|---|
| Batch | Product | treatment (%) | treatment (%) |
| S024-J01-13A | S706 | 5.2 | 2.0 |
| S024-J21-13A | S706 | 4.5 | 3.2 |

As can be seen from the results in Table 10, the solutions of reduced astringency soy protein product were substantially clear before heat treatment and the level of haze was actually reduced by the heat treatment.

### Example 8

This Example describes the production of soy protein products according to the methods of the aforementioned U.S. Pat. Nos. 8,563,071 and 8,691,318 and U.S. patent application Ser. No. 13/879,418 filed Aug. 1, 2013 (US Patent Publication No. 2013-0316069 published Nov. 28, 2013) (“S701”).

‘a’ kg of ‘b’ was combined with ‘c’ L of ‘d’ M CaCl2 solution at ‘e’ and agitated for ‘f’ minutes to provide an aqueous protein solution. The bulk of the residual solids were removed and the resulting protein solution was partially clarified by centrifugation with a decanter centrifuge. The sample was further clarified by centrifugation with a disc stack centrifuge to provide ‘h’ L of centrate having a protein content of ‘i’ % by weight. The sample was additionally clarified by filtration to provide ‘j’ L of protein solution having a protein content of ‘k’ % by weight.

‘l’ L of clarified protein solution was then added to ‘m’ L of RO purified water and the pH of the sample lowered to ‘n’ with diluted HCl.

The diluted and acidified protein extract solution was reduced in volume from ‘o’ L to ‘p’ L by concentration on a polyethersulfone (PES) membrane having a molecular weight cut-off of ‘q’ daltons, operated at a temperature of about ‘r’ ° C. The acidified protein solution, with a protein content of ‘s’ wt %, was diafiltered with ‘t’ L of RO purified water, with the diafiltration operation conducted at about ‘u’ ° C. The resulting diafiltered protein solution was then ‘v’. The concentrated and diafiltered protein solution, having a protein content of ‘w’ % by weight, represented a yield of ‘x’ wt % of the initial clarified protein solution. ‘y’ kg of the concentrated and diafiltered protein solution was diluted with ‘z’ L of water then ‘aa’ kg of the sample dried to yield a product found to have a protein content of ‘ab’ % (N×6.25) d.b. The product was given designation ‘ac’ S701.

The parameters ‘a’ to ‘ac’ for two runs are set forth in the following Table 11:

TABLE 11
Parameters for the runs to produce S701

| ac | S005-K18-08A | S024-J07-13A | |
|---|---|---|---|
| a | 60 | 60 | |
| b | defatted, minimally heat | defatted soy white flakes | |
| | processed soy flour | | |
| c | 600 | 600 | |
| d | 0.15 | 0.09 | |
| e | ambient temperature | 60° | C. |
| f | 60 | 30 | |
| h | 463 | 439 | |
| i | 3.59 | 2.73 | |
| j | 410 | not applicable | |
| k | 2.63 | not applicable | |
| l | 410 | 439 | |
| m | 410 | 286 | |
| n | 3.07 | 3.23 | |
| o | 820 | 717 | |
| p | 70 | 217 | |
| q | 10,000 | 100,000 | |
| r | 29 | 51 | |
| s | 11.21 | 4.92 | |
| t | 350 | 326 | |
| u | 29 | 49 | |
| v | not applicable | further concentrated | |
| w | 13.34 | 11.68 | |
| x | 89.6 | 78.0 | |
| y | 36.21 kg | 80 | L |
| z | not applicable | 40 | L |
| aa | 36.21 kg | 41.32 | kg |

| | | |
|---|---|---|
| ab | 102.71 | 99.14 |

### Example 9

This Example illustrates a comparison of the astringency level of the S024-J01-13A S706 prepared as described in Example 1 with that of the S005-K18-08A S701 prepared as described in Example 8.

Two sets of each sample were prepared for sensory evaluation by weighing out sufficient protein powder to supply a certain weight of protein and then dissolving the protein powder in purified drinking water at a ratio of 50 parts water per weight of supplied protein. For one test the pH of the sample of S706 was lowered to the pH of the S701 solution (3.32) by the addition of food grade HCl solution. In the second test the pH of both samples were adjusted to about 3.5 with either food grade HCl or food grade NaOH solution as necessary. An informal panel of seven panellists was asked to blindly taste samples having matched pH values and indicate which was less astringent. The evaluation was then repeated with a second set of samples. Results from the two trials were combined.

Seven out of 14 panellists indicated that the S024-J01-13A S706 was less astringent, six panellists indicated that the S005-K18-08A S701 was less astringent and one panellist could not detect a difference in the astringency of the samples.

### Example 10

This Example illustrates a comparison of the astringency level of the S024-J21-13A S706 prepared as described in Example 1 with that of the S005-K18-08A S701 prepared as described in Example 8.

Samples were prepared for sensory evaluation by weighing out sufficient protein powder to supply a certain weight of protein and then dissolving the protein powder in purified drinking water at a ratio of 50 parts water per weight of supplied protein. The pH of both samples was adjusted to about 3.5 with either food grade HCl or food grade NaOH solution as necessary. An informal panel of seven panellists was asked to blindly taste the samples and indicate which was less astringent.

Six out of seven panellists indicated that the S024-J21-13A S706 was less astringent and one panellist identified the S005-K18-08A S701 as less astringent.

### Example 11

This Example illustrates a comparison of the astringency level of the S024-J21-13A S706 prepared as described in Example 1 with that of the S024-J07-13A S701 prepared as described in Example 8.

Samples were prepared for sensory evaluation by weighing out sufficient protein powder to supply a certain weight of protein and then dissolving the protein powder in purified drinking water at a ratio of 50 parts water per weight of supplied protein. The pH of the S024-J07-13A S701 sample was 3.49. The pH of the S024-J21-13A S706 sample was adjusted from 3.85 to 3.49 with food grade HCl solution. An informal panel of eight panellists was asked to blindly taste the samples and indicate which was less astringent.

Five out of eight panellists indicated that the S024-J21-13A S706 was less astringent, two panellists identified the S024-J07-13A S701 as less astringent, while one panellist could not identify which sample was less astringent.

### Example 12

This Example illustrates a comparison of the astringency level of the S024-F12-14A S706 prepared as described in Example 1 with that of the S024-J07-13A S701 prepared as described in Example 8.

Two sets of each sample were prepared for sensory evaluation by weighing out sufficient protein powder to supply a certain weight of protein and then dissolving the protein powder in purified drinking water at a ratio of 50 parts water per weight of supplied protein. The pH of the S706 samples was about 3.7 and HCl solution was added to lower the pH to match that of the S701 samples (about 3.5). An informal panel of seven panellists was asked to blindly taste one set of S706 and S701 samples and indicate which was less astringent. The evaluation was then repeated with six panellists and a second set of samples. Results from the two trials were combined.

Seven out of 13 panellists indicated that the S024-F12-14A S706 was less astringent, five panellists indicated that the S005-J07-13A S701 was less astringent and one panellist could not detect a difference in the astringency of the samples.

### Example 13

This Example contains an evaluation of the dry colour and colour in solution of the co-products (S706B) of the production of reduced astringency soy protein products, prepared according to the method of Example 1.

The colour of the dry powders was assessed using a HunterLab ColorQuest XE instrument in reflectance mode. The colour values are set forth in the following Table 12:

TABLE 12
HunterLab scores for dry protein products

| Sample | L* | a* | b* |
|---|---|---|---|
| S024-J01-13A S706B | 85.37 | −0.51 | 12.03 |
| S024-J21-13A S706B | 87.40 | −1.31 | 10.26 |
| S024-F11-14A S706B | 88.57 | −0.33 | 8.63 |
| S024-F12-14A S706B | 86.31 | −1.19 | 11.37 |

As may be seen from the results in Table 12, the co-products that were adjusted in pH were darker than the reduced astringency soy protein products, while there was little difference in L* value when the co-product was not adjusted in pH. All of the co-products prepared were greener and more yellow than the reduced astringency soy protein products.

Solutions of the co-products from the preparation of reduced astringency soy protein products were prepared by dissolving sufficient protein powder to supply 0.48 g of protein in 15 ml of RO purified water. The pH of the solutions was measured with a pH meter and the colour and clarity assessed using a HunterLab ColorQuest XE instrument operated in transmission mode. The results are shown in the following Table 13.

TABLE 13
pH and HunterLab scores for solutions of soy protein products

| sample | pH | L* | a* | b* | haze |
|---|---|---|---|---|---|
| S024-J01-13A S706B | 6.54 | 48.61 | 1.86 | 22.39 | 95.3 |
| S024-J21-13A S706B | 7.16 | 67.43 | 1.00 | 17.26 | 97.2 |
| S024-F11-14A S706B | 3.28 | 97.61 | −0.89 | 7.32 | 5.9 |
| S024-F12-14A S706B | 7.65 | 64.42 | 0.45 | 16.36 | 96.5 |

As may be seen from the results in Table 13, the solution properties of the co-products of the production of the reduced astringency soy protein products were influenced by the pH of the samples. The co-product sample that was not raised in pH gave a much clearer solution that was lighter, greener and bluer in colour, being quite similar in properties to the solutions of the reduced astringency soy products.

### Example 14

This Example contains an evaluation of the solubility in water of the co-products of the production of the reduced astringency soy products, prepared by the methods of Example 1. Solubility was tested based on protein solubility (termed protein method), a modified version of the procedure of Morr et al., J. Food Sci. 50:1715-1718).

Sufficient protein powder to supply 0.5 g of protein was weighed into a beaker and then a small amount of RO purified water was added and the mixture stirred until a smooth paste formed. Additional water was then added to bring the volume to approximately 45 ml. The contents of the beaker were then slowly stirred for 60 minutes using a magnetic stirrer. The pH was determined immediately after dispersing the protein and was adjusted to the appropriate level (2, 3, 4, 5, 6 or 7) with diluted NaOH or HCl. The pH was then measured and corrected periodically during the 60 minutes stirring. After the 60 minutes of stirring, the samples were made up to 50 ml total volume with RO purified water, yielding a 1% w/v protein dispersion. The protein content of the dispersions was determined by combustion analysis using a Leco Nitrogen Determinator. The samples were then centrifuged at 7,800 g for 10 minutes, which sedimented insoluble material and yielded a supernatant. The protein content of the supernatant was measured by combustion analysis.

Solubility of the product was then calculated:

Solubility(protein method)(%)=(% protein in supernatant/% protein in initial dispersion)×100 1)

Values calculated as greater than 100% were reported as 100%.

The solubility results obtained are set forth in the following Table 14:

TABLE 14
Solubility of products at different
pH values based on protein method

| | | Solubility (protein method) (%) | | | | | |
|---|---|---|---|---|---|---|---|
| Batch | Product | pH 2 | pH 3 | pH 4 | pH 5 | pH 6 | pH 7 |
| S024-J01-13A | S706B | 17.2 | 16.8 | 10.5 | 2.0 | 0.0 | 0.0 |
| S024-J21-13A | S706B | 53.9 | 47.0 | 24.7 | 5.7 | 10.5 | 17.3 |

As may be seen from the results in Table 14, the pH adjusted co-products of the production of the reduced astringency soy protein products were generally low in solubility, particularly over the pH range of 5 to 7.

### Example 15

This Example contains an evaluation of the water binding capacity of the co-products of the production of the reduced astringency soy products, prepared by the methods of Example 1.

Protein powder (1 g) was weighed into centrifuge tubes (50 ml) of known weight. To this powder was added approximately 20 ml of RO water at the natural pH. The contents of the tubes were mixed using a vortex mixer at moderate speed for 1 minute. The samples were incubated at room temperature for 5 minutes then mixed with the vortex mixer for 30 seconds. This was followed by incubation at room temperature for another 5 minutes followed by another 30 seconds of vortex mixing. The samples were then centrifuged at 1,000 g for 15 minutes at 20° C. After centrifugation, the supernatant was carefully poured off, ensuring that all solid material remained in the tube. The centrifuge tube was then re-weighed and the weight of water saturated sample was determined

Water binding capacity (WBC) was calculated as:

WBC(ml/g)=(mass of water saturated sample−mass of initial sample)/(mass of initial sample×total solids content of sample)

The water binding capacity results obtained are set forth in the following Table 15.

TABLE 15
Water binding capacity of various products

| product | WBC (ml/g) |
|---|---|
| S024-J01-13A S706B | 4.48 |
| S024-J21-13A S706B | 7.27 |

As may be seen from the results of Table 15, the pH adjusted co-products of the production of the reduced astringency soy protein products had good water binding capacities.

### Example 16

This Example illustrates the preparation of a soy protein isolate by conventional isoelectric precipitation (MP).

30 kg of soy white flake was added to 300 L of RO purified water at ambient temperature and the pH adjusted to 8.5 by the addition of 1M sodium hydroxide solution. The sample was agitated for 30 minutes to provide an aqueous protein solution. The pH of the extraction was monitored and maintained at 8.5 throughout the 30 minutes. The residual soy white flake was removed and the resulting protein solution clarified by centrifugation and filtration to produce 278.7 L of filtered protein solution having a protein content of 2.93% by weight. The pH of the protein solution was adjusted to 4.5 by the addition of HCl that had been diluted with an equal volume of water and a precipitate formed. The precipitate was collected by centrifugation then washed by re-suspending it in 2 volumes of RO purified water. The washed precipitate was then collected by centrifugation. A total of 32.42 kg of washed precipitate was obtained with a protein content of 18.15 wt %. This represented a yield of 72.0% of the protein in the clarified extract solution. An aliquot of 16.64 kg of the washed precipitate was combined with an equal weight of RO purified water and then the pH of the sample adjusted to 6 with sodium hydroxide. The pH adjusted sample was then spray dried to yield an isolate with a protein content of 93.80% (N×6.25) d.b. The product was designated S013-K19-09A conventional IEP pH 6.

### Example 17

This Example is a sensory evaluation of the S024-J01-13A S706B product prepared as described in Example 1 with the conventional soy protein isolate product prepared as described in Example 14.

Samples were prepared for sensory evaluation by weighing out sufficient protein powder to supply a certain weight of protein and then dissolving the protein powder in purified drinking water at a ratio of 50 parts water per weight of supplied protein. The pH of the S706B solution was found to be 6.64. The initial pH of the S013-K19-09A conventional IEP pH 6 sample was 5.49 and it was adjusted to 6.66 with food grade sodium hydroxide solution. An informal panel of nine panellists was asked to blindly taste the samples and indicate which had less beany flavour.

Eight out of nine panellists found the S024-J01-13A S706B sample to have less beany flavour while the ninth panellist could not identify one sample as less beany.

### Example 18

This Example is a sensory evaluation of the S024-J21-13A S706B product prepared as described in Example 1 with the conventional soy protein isolate product prepared as described in Example 14.

Samples were prepared for sensory evaluation by weighing out sufficient protein powder to supply a certain weight of protein and then dissolving the protein powder in purified drinking water at a ratio of 50 parts water per weight of supplied protein. The pH of the S706B solution was found to be 7.13. The initial pH of the S013-K19-09A conventional IEP pH 6 sample was 5.45 and it was adjusted to 7.18 with food grade sodium hydroxide solution. An informal panel of seven panellists was asked to blindly taste the samples and indicate which had less beany flavour.

Six out of seven panellists found the S024-J21-13A S706B sample to have less beany flavour while the seventh panellist could not identify one sample as less beany.

### Example 19

This Example illustrates the molecular weight profile of the soy protein products of the present invention, prepared as described in Example 1 and the commercial soy protein products Pro Fam 825 and Pro Fam 875 (both ADM, Decatur, IL).

Molecular weight profiles were determined by size exclusion chromatography using a Varian ProStar HPLC system equipped with a 300×7.8 mm Phenomenex BioSep S-2000 series column. The column contained hydrophilic bonded silica rigid support media, 5 micron diameter, with 145 Angstrom pore size.

Before the soy protein samples were analyzed, a standard curve was prepared using a Biorad protein standard (Biorad product #151-1901) containing proteins with known molecular weights between 17,000 Daltons (myoglobulin) and 670,000 Daltons (thyroglobulin) with Vitamin B12 added as a low molecular weight marker at 1,350 Daltons. A 0.9% w/v solution of the protein standard was prepared in water, filtered with a 0.45 μm pore size filter disc then a 50 μL aliquot run on the column using a mobile phase of 0.05M phosphate/0.15M NaCl, pH 6 containing 0.02% sodium azide. The mobile phase flow rate was 1 mL/min and components were detected based on absorbance at 280 nm. Based on the retention times of these molecules of known molecular weight, a regression formula was developed relating the natural log of the molecular weight to the retention time in minutes.

Retention time(min)=−0.955×In(molecular weight)+18.502(r²=0.999)

For the analysis of the soy protein samples, 0.05M NaCl, pH 3.5 containing 0.02% sodium azide was used as the mobile phase and also to dissolve dry samples. Protein samples were mixed with mobile phase solution to a concentration of 1% w/v, placed on a shaker for at least 1 hour then filtered using 0.45 μm pore size filter discs. Sample injection size was 50 μL. The mobile phase flow rate was 1 mL/minute and components were detected based on absorbance at 280 nm.

The above regression formula relating molecular weight and retention time was used to calculate retention times that corresponded to molecular weights of 100,000 Da, 15,000 Da, 5,000 Da and 1,000 Da. The HPLC ProStar system was used to calculate the peak areas lying within these retention time ranges and the percentage of protein ((range peak area/total protein peak area)×100) falling in a given molecular weight range was calculated. Note that the data was not corrected by protein response factor.

The molecular weight profiles of the products prepared as described in Examples 1 and 8 and the commercial products are shown in Table 16.

TABLE 16
Molecular weight profile of soy protein products

| | % >100,000 | % 15,000-100,000 | % 5,000-15,000 | % 1,000-5,000 |
|---|---|---|---|---|
| product | Da | Da | Da | Da |
| S024-J01-13A S706 | 59.1 | 33.8 | 4.8 | 2.3 |
| S024-J21-13A S706 | 72.8 | 22.7 | 2.9 | 1.7 |
| S024-F11-14A S706 | 37.1 | 25.6 | 25.9 | 11.3 |
| S024-F12-14A S706 | 41.7 | 23.6 | 24.1 | 10.5 |
| S024-J01-13A S706B | 37.2 | 23.4 | 5.2 | 34.3 |
| S024-J21-13A S706B | 40.1 | 28.5 | 5.1 | 26.3 |
| S024-F11-14A S706B | 94.8 | 3.3 | 0.7 | 1.3 |
| S024-F12-14A S706B | 41.3 | 24.2 | 6.1 | 28.3 |
| Pro Fam 825 | 3.2 | 30.2 | 32.5 | 34.1 |
| Pro Fam 875 | 0.5 | 19.6 | 33.7 | 46.2 |

As may be seen from the results presented in Table 16, the molecular weight profiles of the products prepared according to Example 1 were different from the molecular weight profiles of the commercial soy protein products. The molecular weight profile of the S706B appeared to be sensitive to the pH at which the final product was prepared. The effect of the pH difference on the proteins may have resulted in differing solubilities in the running buffer used to prepare the samples for HPLC analysis.

### Example 20

This Example is another illustration of the molecular weight profile of the soy protein products of the present invention, prepared as described in Example 1 and the commercial soy protein products Pro Fam 825 and Pro Fam 875 (both ADM, Decatur, IL).

Molecular weight profiles were determined by size exclusion chromatography using a Varian ProStar HPLC system equipped with a 300×7.8 mm Phenomenex BioSep S-2000 series column. The column contained hydrophilic bonded silica rigid support media, 5 micron diameter, with 145 Angstrom pore size.

Before the soy protein samples were analyzed, a standard curve was prepared using a Biorad protein standard (Biorad product #151-1901) containing proteins with known molecular weights between 17,000 Daltons (myoglobulin) and 670,000 Daltons (thyroglobulin) with Vitamin B12 added as a low molecular weight marker at 1,350 Daltons. A 0.9% w/v solution of the protein standard was prepared in water, filtered with a 0.45 μm pore size filter disc then a 50 μL aliquot run on the column using a mobile phase of 0.05M phosphate/0.15M NaCl, pH 6 containing 0.02% sodium azide. The mobile phase flow rate was 1 mL/min and components were detected based on absorbance at 280 nm. Based on the retention times of these molecules of known molecular weight, a regression formula was developed relating the natural log of the molecular weight to the retention time in minutes.

Retention time(min)=−0.865×In(molecular weight)+17.154(r²=0.98)

For the analysis of the soy protein samples, 0.05M phosphate/0.15M NaCl, pH 6 containing 0.02% sodium azide was used as the mobile phase and also to dissolve dry samples. Protein samples were mixed with mobile phase solution to a concentration of 1% w/v, placed on a shaker for at least 1 hour then filtered using 0.45 μm pore size filter discs. Sample injection size was 50 μL. The mobile phase flow rate was 1 mL/minute and components were detected based on absorbance at 280 nm.

The above regression formula relating molecular weight and retention time was used to calculate retention times that corresponded to molecular weights of 100,000 Da, 15,000 Da, 5,000 Da and 1,000 Da. The HPLC ProStar system was used to calculate the peak areas lying within these retention time ranges and the percentage of protein ((range peak area/total protein peak area)×100) falling in a given molecular weight range was calculated. Note that the data was not corrected by protein response factor.

The molecular weight profiles of the products prepared as described in Examples 1 and 8 and the commercial products are shown in Table 17.

TABLE 17
Molecular weight profile of soy protein products

| | % >100,000 | % 15,000-100,000 | % 5,000-15,000 | % 1,000-5,000 |
|---|---|---|---|---|
| product | Da | Da | Da | Da |
| S024-J01-13A S706 | 21.6 | 56.3 | 13.3 | 8.7 |
| S024-J21-13A S706 | 28.3 | 50.1 | 11.0 | 10.6 |
| S024-F11-14A S706 | 12.3 | 51.6 | 22.5 | 13.6 |
| S024-F12-14A S706 | 14.5 | 50.4 | 21.7 | 13.4 |
| S024-J01-13A S706B | 31.9 | 18.6 | 5.6 | 43.8 |
| S024-J21-13A S706B | 32.6 | 19.9 | 5.5 | 41.9 |
| S024-F11-14A S706B | 15.9 | 21.1 | 6.6 | 56.4 |
| S024-F12-14A S706B | 23.5 | 21.2 | 6.4 | 48.9 |
| Pro Fam 825 | 36.2 | 30.8 | 17.3 | 15.6 |
| Pro Fam 875 | 26.3 | 30.1 | 21.5 | 22.0 |

As may be seen from the results presented in Table 17, the molecular weight profiles of the products prepared according to Example 1 were different from the molecular weight profiles of the commercial soy protein products.

### Example 21

This Example contains an evaluation of the phytic acid content of the soy protein products produced as described in Example 1. Phytic acid content was determined using the method of Latta and Eskin (J. Agric. Food Chem., 28: 1313-1315).

The results obtained are set forth in the following Table 17.

TABLE 18
Phytic acid content of protein products

| product | % phytic acid d.b. |
|---|---|
| S024-J01-13A S706 | 0.00 |
| S024-J21-13A S706 | 0.00 |
| S024-F11-14A S706 | 0.09 |
| S024-F12-14A S706 | 0.05 |
| S024-J01-13A S706B | 0.01 |
| S024-J21-13A S706B | 0.00 |
| S024-F11-14A S706B | 0.09 |
| S024-F12-14A S706B | 0.00 |

As may be seen from the results in Table 18, all of the products tested had either low or undetectable levels of phytic acid.

### SUMMARY OF THE DISCLOSURE

In summary of this disclosure, the present invention provides soy protein products, preferably soy protein isolates, which have reduced astringency when tasted in an acidic solution such as an acidic beverage. Modifications are possible within the scope of the present invention.

1. A method of preparing soy protein product with reduced astringency when tasted in aqueous solution at a pH below about 5, which comprises:
(a) extracting a soy protein source with an aqueous calcium salt solution to cause solubilization of soy protein from the protein source and to form an aqueous soy protein solution,
(b) separating the aqueous soy protein solution from residual soy protein source,
(c) optionally diluting the aqueous soy protein solution,
(d) adjusting the pH of the aqueous soy protein solution to a pH of about 1.5 to about 4.4 to produce an acidified soy protein solution,
(e) optionally clarifying the acidified soy protein solution if it is not already clear,
(f) alternatively from steps (b) to (e), optionally, diluting and then adjusting the pH of the combined aqueous soy protein solution and residual soy protein source to a pH of about 1.5 to about 4.4 and then separating the acidified, preferably clear, soy protein solution from residual soy protein source, and
(g) fractionating the proteins in the acidified soy protein solution of step (e) or (f) to separate lower molecular weight, less astringent proteins from higher molecular weight, more astringent proteins, wherein said fractionation step is effected by membrane processing the acidified aqueous soy protein solution fractionate the protein components of the acidified aqueous soy protein solution into a higher molecular weight fraction in a first retentate and a lower molecular weight fraction in a first permeate,
(h) membrane processing the first permeate to retain the lower molecular weight fraction protein components in a second retentate and to permit contaminants to pass the membrane in a second permeate,
(i) optionally drying the second retentate to provide a soy protein product of reduced astringency.

2-26. (canceled)

27. A soy protein product having a protein content of at least about 60% wt % (N×6.25) d.b. and which
is completely soluble in aqueous media at acid pH values of less than about 4.4;
is heat stable in aqueous media at acid values of less than about 4.4;
does not require stabilizers or other additives to maintain the protein product in solution or suspension;
is low in phytic acid; and
is low in astringency when tasted in aqueous solution at a pH below about 5.

28-34. (canceled)

35. A soy protein product having a protein content of at least about 60 wt % (N×6.25) d.b. and having low astringency when tasted in aqueous solution at a pH of below about 5 which is substantially completely soluble in an aqueous medium at a pH of less than about 4.4.

36-86. (canceled)

87. A soy protein product having a molecular weight profile which is selected from the group consisting of:
(A) about 32 to about 78% greater than about 100,000 Da;
about 18 to about 48% from about 15,000 to about 100,000 Da;
about 0 to about 31% from about 5,000 to about 15,000 Da; and
about 0 to about 17% from about 1,000 to about 5,000 Da.
(B) about 32 to about 100% greater than about 100,000 Da;
about 0 to about 34% from about 15,000 to about 100,000 Da;
about 0 to about 12% from about 5,000 to about 15,000 Da; and
about 0 to about 40% from about 1,000 to about 5,000 Da
(C) about 7 to about 35% greater than about 100,000 Da;
about 21 to about 62% from about 15,000 to about 100,000 Da;
about 6 to about 28% from about 5,000 to about 15,000 Da; and
about 3 to about 28% from about 1,000 to about 5,000 Da and having a phytic acid content of less than about 1.5 wt %; and
(D) about 10 to about 38% greater than about 100,000 Da;
about 13 to about 33% from about 15,000 to about 100,000 Da;
about 0 to about 12% from about 5,000 to about 15,000 Da; and
about 36 to about 62% from about 1,000 to about 5,000 Da.
